(19) United States
(12) Patent Application Publication
(10) Pub. No.: US 2015/0030989 A1
(43) Pub. Date: Jan. 29, 2015

(54) Handheld Device for Delivering Photodynamic Therapy

(71) Applicants: Nikolaos S. Soukos, Saugus, MA (US); Philip P. Stashenko, Medfield, MA (US)

(72) Inventors: Nikolaos S. Soukos, Saugus, MA (US); Philip P. Stashenko, Medfield, MA (US)

(73) Assignee: The Forsyth Institute, Cambridge, MA (US)

(21) Appl. No.: 14/482,722

(22) Filed: Sep. 10, 2014

Related U.S. Application Data
(63) Continuation of application No. PCT/US13/32348, filed on Mar. 15, 2013.
(60) Provisional application No. 61/647,827, filed on May 16, 2012.

(51) Int. Cl. A61N 5/06; A61C 19/06

(52) U.S. Cl. CPC A61N 5/062; A61C 19/063; A61N 5/0624; A61N 2005/063; A61N 2005/0644
USPC 433/29; 433/217.1

(57) ABSTRACT

The present invention relates to a handheld dental device that provides photodynamic therapy. The device of the present invention may have an elongated housing; an applicator tip extending from the distal end of the housing; wherein the applicator tip has a lumen defined by a wall; an optic fiber extending through the lumen of the applicator tip; at least one power source attached to or residing within the housing; at least one light source that provides light to the optic fiber and emits a wavelength that ranges from about 650 nm to about 1400 nm; and at least one oxygen source that provides oxygen to a passage that communicates with the oxygen source and the applicator tip. Oxygen and light may be provided at the applicator tip simultaneously. The device may be used to eliminate or reduce bacteria which cause oral disease or conditions, such as endodontic treatment failures.

### CROSS-REFERENCE TO RELATED APPLICATIONS

This application claims the benefit of under 35 U.S.C. §365(c) of International Patent Application No. PCT/US2013/032348, filed on Mar. 15, 2013, which claims the benefit of U.S. Provisional Application No. 61/647,827, filed on May 16, 2012; the entire contents of said applications are incorporated by reference herein in their entirety.

### GOVERNMENT SUPPORT

The invention was supported, at least in part, by a grant RO1-DE-16922 from the National Institute of Dental and Craniofacial Research (NIDCR). The Government has certain rights in the invention.

### BACKGROUND

One of the goals of endodontic treatment is the elimination of bacteria from the dental root canal system. However, complete disinfection of the root canal system has been difficult to achieve by standard endodontic chemo-mechanical debridement despite recent technological advances such as rotary instrumentation and chemical irrigants. This is primarily due to the complex anatomy of the root canal system, particularly the dentinal tubules, into which bacteria penetrate, remain viable and can re-infect teeth resulting in treatment failure. In addition, current endodontic procedures require very good technical skills, and use medicaments whose effectiveness has not, for the most part, been definitively proven in human clinical trials. Systematic reviews demonstrate that endodontic failure rates are about 22-25%.

Studies show that infected teeth have a 10-15% lower success rate than minimally infected teeth. Given that more than 20 million root canals are performed yearly in the U.S., more than 2 million endodontic re-treatments, which often involve surgery, could be avoided by better disinfection procedures. These numbers are indicative of the health, social and economic consequences of failures of root canal treatments. The cumulative economic impact is on the order of billions of dollars. Hence, a need exists for the development of an adjunctive antimicrobial device-procedure to standard chemo-mechanical debridement that improves outcomes for endodontic treatment by effectively eliminating microorganisms in the root canal system (root canal(s) and dentinal tubules). A need also exists to eliminate bacteria in any target area of the oral cavity.

### SUMMARY

This description is not to be taken in a limiting sense, but is made merely for the purpose of illustrating the general principles of the invention. The section titles and overall organization of the present detailed description are for the purpose of convenience only and are not intended to limit the present invention.

The present invention relates to a handheld dental device that provides photodynamic therapy that can be used to effectively eliminate bacteria in the oral cavity. The device of the present invention may include an elongated housing having a distal end and a proximal end; an applicator tip extending from the distal end of the housing; wherein the applicator tip has a lumen defined by a wall; an optic fiber extending from the distal end of the housing through the lumen of the applicator tip; at least one power source attached to or residing within the housing, wherein the power source is in communication with at least the light source; at least one light source within or at the housing, wherein the light source provides light to the optic fiber; and at least one oxygen source within or at the housing, wherein the oxygen source provides oxygen to a passage that communicates with the oxygen source and the applicator tip; and wherein oxygen and light are provided at the applicator tip. The device may further include, for example, a switch that communicates with the light source, the passage of oxygen, or both. In an embodiment, oxygen and light are provided at the applicator tip simultaneously or sequentially in time. The light source can emit a wavelength that ranges from about 650 nm to about 1400 nm, a power density that ranges from about 1 mW/cm2 to about 200 mW/cm2 and/or an energy fluence that ranges from about 0.1 Joules/cm2 to about 1000 Joules/cm2. In yet another embodiment, the optic fiber may comprise at least one diffuser for distributing light (e.g., in essentially 360 degrees). The present invention may embody an optic fiber has a diameter ranging from about 100 μm to about 500 μm, and/or a length ranging from about 5 mm to about 40 mm. The applicator tip can have a plurality of pores through which oxygen flows, and in an aspect can be flexible (e.g., bends in a range from about 5 degrees to about 90 degrees), interchangeable or both. The applicator tip can have various lengths including a length ranging from about 5 mm to 40 mm. The oxygen can flow from the applicator tip at a rate between e.g., about 1 ml/min and about 10 ml/min.

The present invention may also embody methods for using the handheld dental device described herein to provide photodynamic therapy. The methods may involve applying at least one photosensitizer to the target area and engaging the handheld device to administer light and oxygen to the target area of the oral cavity of the individual.

Methods for improving oral health in an individual are encompassed by the present invention. The methods involve administering light and oxygen to the target area of the oral cavity of the individual using the handheld device described herein; wherein a reduction in one or more of the following occurs: a number of one or more bacteria; a number of one or more fungi; the severity or number of endodontic treatment failures; and at least one symptom associated with one or more gum conditions or diseases; as compared to that not subjected to light and oxygen administration.

The present invention may further include a handheld dental system or kit for delivering photodynamic therapy. In some embodiments, the system or kit has the handheld dental device that comprises an elongated housing having a distal end and a proximal end; an optic fiber extending from the distal end of the housing through the lumen of the applicator tip; and at least one light source within or at the housing, wherein the light source provides light to the optic fiber. The system or kit may also include an applicator tip for attachment to the distal end of the housing; wherein the applicator tip has a lumen defined by a wall; at least one power source for attachment or insertion to the housing, wherein the power source is in communication with at least the light source; at least one oxygen source attachment or insertion to the housing, wherein the oxygen source provides oxygen to a passage that communicates with the oxygen source and the applicator tip; and at least one photosensitizer (e.g., methylene blue, acridine orange, porfimer sodium, benzoporphyrin derivative, methoxsalen, psoralen, talaporfin, temoporfin, verteporfin or a combination thereof).

There are numerous advantages of the present invention. The device of the present invention may allow chair-side application of photodynamic therapy using a handheld device. This easy and efficient use of photodynamic therapy following standard chemo-mechanical debridement will result in less endodontic treatment failures as well as better treatment of other oral infectious disease. In particular, a use of the device of the present invention in endodontic treatment may include: rapid bacterial killing due to short exposure to light emitted by the device following application of a photosensitizing compound in the root canal; enhancement of bacterial killing with the simultaneous release of light and oxygen from the device; full penetration of oxygen and light into biofilms and within dentinal tubules; limited toxicity of light on both periodontal ligament and adjacent bone; and absence of thermal side effects in the tissues surrounding the roots. In some embodiments, the device and methods of the present invention offer the following clinical advantages: a) ease of use; b) rapid bacterial killing in the entire root canal system; and c) limited toxicity in the tissues surrounding the roots. Additionally, patients with highly infected cases with accompanying radiographic findings often require multiple appointments to allow disinfecting medicaments to act on an inter-appointment basis. The device and methods of the present invention may be used to eliminate the need for patients to have multiple appointments, which translates into saving time and money, increasing productivity, and providing a better experience for the patient.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic of the photodynamic therapy of one embodiment of the present invention.

FIGS. 2A & 2B are photographs of the optic fiber used in one embodiment of the present invention.

FIG. 3 are photographs of a side view without an applicator (FIG. 3(1)), front view (FIG. 3(2)), a side view without the applicator but with the optic fiber (FIG. 3(3)), and a side view with the applicator tip (FIG. 3(5)).

FIG. 3A is a photograph of a side view with a larger applicator tip for vital pulp therapy.

FIGS. 4A-4B are photographs of scanning electron microscopy showing multispecies biofilms developed on the root canal surface (A) and invasion of microorganisms in dentinal tubules (B) 3 days following infection with the microorganisms.

FIG. 5 is a bar graph showing phototoxicity of multispecies root canal biofilms after incubation with 25 μg/ml methylene blue (MB) for 10 minutes followed by treatment with red light of 665 nm (30 J/cm²) and colony forming units (CFU) assay.

FIG. 6 is a series of Confocal Scanning Laser Microscopy images (X-Z) obtained from the mid-root area of canals.

FIG. 7 presents two bar graphs on the growth of methylene blue (MB)-sensitized gingival fibroblasts and osteoblasts as determined by the MTT assay after their exposure to red light.

FIG. 8A-8B presents Western blots obtained from osteoblasts (A) and fibroblasts (B) following their treatment with methylene blue (MB) only (lane MB+), light only (lane L+), or light and MB (lane L+MB+).

FIG. 9 presents the pre- and post-treatment detection frequencies for 39 species found in endodontic infections by checkerboard DNA-DNA hybridization with whole genomic probes for 45 canals treated by chemomechanical debridement (CMD)+Photodynamic Therapy (PDT) and 44 treated by CMD alone. (⋄ CMD, baseline, ♦ post-CMD, ◯ PDT, baseline, post-PDT).

### DETAILED DESCRIPTION

The present invention relates to a handheld dental device for delivering photodynamic therapy (PDT). PDT generally involves three components: light, oxygen and a photosensitizer (PS). The device of the present invention may provide at least two of these components, namely, light and oxygen. In an embodiment, the third component, a photosensitizing agent can be applied by the user (e.g., a dentist or dental professional), or alternatively, the device can be modified to include a compartment for storing and applying the photosensitizer at the desired time.

The device of the present invention may be used to reduce or eliminate bacteria/fungi that cause oral disease or conditions. In particular, the device of the present invention can be used in providing endodontic treatment of root canals to reduce the incidence of re-infection of the root canal or failure of the endodontic treatment. In particular, the present invention can further be used to disinfect the surgical site (e.g., bony crypt) during procedures. Additionally, the present invention can be used with endodontic regenerative procedures including the use of stem or pluripotent cells. The device of the present invention may be used to treat any oral condition associated with bacterial growth or infection. Other oral conditions that can be treated include, for example, periodontal diseases (gingivitis, periodontitis), caries, peri-implantitis, halitosis, and oral candidiasis. Targeted areas of the oral cavity include, in an embodiment, areas of the oral cavity such as root canals, dentinal tubules, dental pulp, tongue, teeth, gums, lingual surfaces, buccal surfaces, palatal surfaces, or facial surfaces, or any combination thereof.

### Housing:

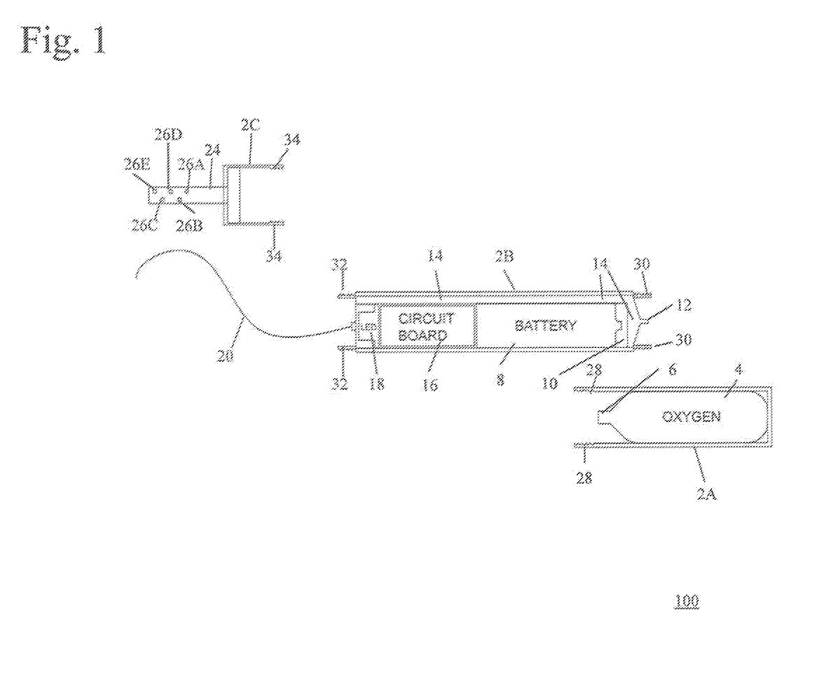

Referring to FIG. 1, handheld device 100 has a housing that is divided into three compartments, back housing 2A, middle housing 2B and front housing 2C. The housing of the handheld device is an elongated cylindrical tube, shaped similarly to a pen. The shape allows the user/dentist to easily hold the device in his/her hand and engage the oxygen and light source to treat the targeted area in an individual's oral cavity. The shape of the housing can be of any shape so long as the device can be manipulated to treat the target tissue. The housing, in an embodiment, can be ergonomically shaped to comfortably fit in the hand of a dental professional (e.g., have indentations and projections to comfortably receive fingers of the user). The shape of the housing can be cylindrical, a rectangular prism, pyramidal, or irregularly shaped.

The housing used in the present invention can be a single compartment or multiple compartments. In the case in which the handheld device is meant to be intended to have replaceable parts, as shown in FIG. 1, then the housing can have more than one detachable/re-attachable compartment (e.g., for replacing the oxygen supply, or a battery). In yet another embodiment, the housing can be a single compartment, but have openings to replace parts or components.

For handheld device 100, the three housing compartments are attached by screw threads 28, 30, 32 and 34. Any mechanism for connecting the housing compartments that are known in the art or developed in the future can be used and include e.g., snaps, latches, clips, screws, and the like.

The housing can be made of any material suitable for housing the components described herein. Preferably, the material is easy to clean, non-flammable, heat resistant and/or bacterial resistant. Examples of such material include stainless steel, ceramic, alloys, plastic, or suitable material. Materials known in the art or developed in the future so long as the material can be formed to house the components described herein.

### Light Source

Handheld device 100 includes light emitting diode 18 and fiber 20 to emit light at certain wavelengths and power densities. A light emitting diode is a light source that provides light sufficient to reduce the number of bacteria at the targeted area along with the application of oxygen and a photosensitizer. In an embodiment, the light sources includes one or more sources that emit light at a wavelength ranging from about 650 nm to about 1400 nm, and/or a power density that ranges from about 1 mW/cm² to about 1000 mW/cm².

In addition to Light Emitting Diodes (LED), light sources of the present invention may also include e.g., gas plasma, linear flash lamps, tungsten halogen, metal halide, Xenon short arc, Mercury short arc, Mercury Xenon short arc, Argon plasma arc, or Argon short arc lamps. The light energy can also be provided by an array of light emitting diodes or laser diodes of suitable wavelength and sufficient power. The light energy can also be provided by chemiluminescent or electroluminescent means. Other light sources are described in U.S. Pat. No. 6,416,319 and PCT WO 2001/026576, which are incorporated by reference. In an embodiment, a diode laser (BWTEK Inc., Newark, Del.) may be used to provide light to the fiber.

For example, light sources also include light radiation sources such as solid-state lighting (SSL) including a light emitting diode (LED) and LED variations, such as, edge emitting LED (EELED), surface emitting LED (SELED) or high brightness LED (HBLED). The LED can be based on different materials such as AlInGaN/AlN, SiC, AlInGaN, GaAs, AlGaAs, GaN, InGaN, AlGaN, AlInGaN, BaN, InBaN, AlGaInP (emitting in NIR and IR), etc. LEDs also include organic LEDs which are constructed with a polymer as the active material and which have a broad spectrum of emission.

Also, light sources include superluminescent diode (SLD) or LED which preferably can provide a broad emission spectrum source. In addition, laser diode (LD), waveguide laser diode (WGLD), and a vertical cavity surface emitting laser (VCSEL) can also be utilized. The same materials used for LED's can be used for diode lasers. Other possibilities include a fiber laser (FL) with laser diode pumping. Fluorescence solid-state light source (FLS) with electro or light pumping from LD, LED or current/voltage sources can also be the radiation source. The FLS can be an organic fiber with electrical pumping.

Lamps such as incandescent lamps, fluorescent lamps, micro halide lamps or other suitable lamps may also be used with the present invention. A lamp can provide the radiation source for white, red, NIR and IR irradiation. For the 5-100 micron range, quantum cascade lasers (QCL) or far infrared emitting diodes can be used. One skilled in the art will appreciate that a variety of radiation sources can provide the necessary optical radiation for the optical appliance depending on size, power requirements, desired treatment regimen, and combinations thereof.

An LED, a laser diode, or a microlamp can generate unwanted heat energy. To accommodate unwanted waste heat, the light emitting handheld device can include heat transfer and/or cooling mechanisms. For example, the device of the present invention can be at least partially formed of a heat conducting material for dissipating heat generated by the light source. For example, portions of the device can be constructed from a material having high thermal conductivity and/or good heat capacitance and is thermally coupled to the light source to extract heat therefrom. One skilled in the art will appreciate that a variety of materials can provide the necessary heat transfer such as, for example, metals including aluminum, copper or their alloy, ceramic and composite materials such as plastics having high thermally conductive components, such as carbon fiber.

### Fiber:

Fiber 20 is in communication with light emitting diode 18 so that the optical fiber can deliver light to the targeted area. Any fiber can be used so long as it can administer or channel light, as described herein. In an embodiment, the fiber has one or more diffusers that can administer 360 degrees of light.

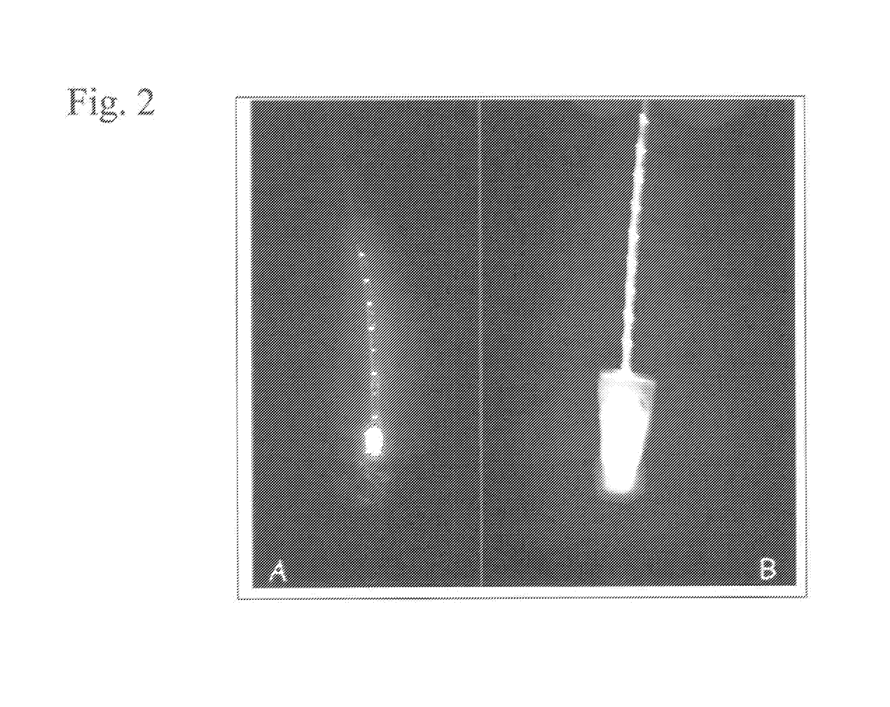

An optical fiber is generally a flexible, transparent fiber made of glass or silica, and acts a waveguide, light channel, or light pipe, to transmit light between the two ends of the fiber. The fiber used in the present invention, in an embodiment, may have one or more diffusers to distribute light. In an aspect, the fiber has multiple cylindrical diffusers that uniformly distributed light at 360°. FIG. 2A shows the optic fiber with a number of diffusers providing 360° of light, which was used to carry out the experiments in the Exemplification. Diffusers can be made by scoring the wall of fiber to allow light to pass through the wall in a 360° fashion. In another embodiment, diffusers can be embedded within the fiber, or made in a continuous fashion when molding the glass/silica into the fiber.

The fiber along with the applicator tip can be angled, bent or curved to conform to the tissue which is being treated with light and oxygen. For example, a root canal naturally has various sizes, curves and shapes. The applicator tip and fiber within it can be inserted into the canal and the flexibility of the tip/fiber conforms to the shape of the canal. This ability to conform to the oral structures of the oral cavity coupled with the delivery of 360° of light allows for efficient and comprehensive light delivery and effective photodynamic therapy to the targeted tissue. Consequently, the applicator tip and the fiber are able to maintain an angle with respect to the housing in this embodiment. In some embodiments, the applicator tip/fiber can bend at angles ranging between about 1 degree to about 90 degrees with respect to the housing.

In particular, using the device, light is emitted to the oral cavity in a wavelength that ranges from about 650 nm to about 1400 nm. In a preferred embodiment, the output is filtered to provide an efficient source of visible red light in the 650-750 nm range or near infrared light in the 750-1400 nm range. In one embodiment, light is filtered to be in the 650-760 nm range. In one embodiment, the light from the light source is not filtered. In one embodiment, the wavelength is 675 nm, 700 nm, 725 nm, 750 nm, 775 nm, 800 nm, 825 nm, 850 nm, 875 nm, 900 nm, 925 nm, 950 nm, 975 nm, 1000 nm, 1025 nm, 1050 nm, 1075 nm, 1100 nm, 1125 nm, 1150 nm, 1175 nm, 1200 nm, 1225 nm, 1250 nm, 1275 nm, 1300 nm, 1325 nm, 1350 nm, 1375 nm, or 1400 nm.

The intensity (energy density) of the light provided by the device of the present invention may range from about 1 mW/cm² to about 1000 mW/cm² or higher, or about 1 mW/cm² to about 800 mW/cm², or from about 1 mW/cm² to about 200 mW/cm², or from about 1 mW/cm² to about 120 mW/cm², or about 20 mW/cm². In another embodiment, the power density, or energy delivered to the teeth, is adjusted to a setting of between about 50 mW/cm² to about 150 mW/cm², or, from about 75 mW/cm² to about 125 mW/cm² (e.g., about 100 mW/cm²).

Additionally, effective amounts of light can be administered in a predetermined dosage. The predetermined dosage may range from about 0.1 Joules/cm² to about 1000 Joules/cm², or from about 0.1 Joules/cm² to about 500 Joules/cm², or, from about 0.1 Joules/cm² to about 100 Joules/cm², or, from about 0.1 Joules/cm² to about 50 Joules/cm², or, from about 0.1 Joules/cm² to about 10 Joules/cm². In one embodiment, the dosage is from about 0.2 Joules/cm² to about 1.2 Joules/cm². In another embodiment, the dosage is about 4.2 Joules/cm². In still another embodiment, the dosage is about 21 Joules/cm². In yet another embodiment, the dosage is 2 Joules/cm², 3 Joules/cm², 4 Joules/cm², 5 Joules/cm², 6 Joules/cm², 7 Joules/cm², 8 Joules/cm², 9 Joules/cm², 10 Joules/cm², 11 Joules/cm², 12 Joules/cm², 13 Joules/cm², 14 Joules/cm², 15 Joules/cm², 16 Joules/cm², 17 Joules/cm², 18 Joules/cm², 19 Joules/cm², 20 Joules/cm², 21 Joules/cm², 22 Joules/cm², 23 Joules/cm², 24 Joules/cm², 25 Joules/cm², 26 Joules/cm², 27 Joules/cm², 28 Joules/cm², 29 Joules/cm², 30 Joules/cm², 31 Joules/cm², 32 Joules/cm², 33 Joules/cm², 34 Joules/cm², 35 Joules/cm², 36 Joules/cm², 37 Joules/cm², 38 Joules/cm², 39 Joules/cm², 40 Joules/cm², 41 Joules/cm², 42 Joules/cm², 43 Joules/cm², 44 Joules/cm², 45 Joules/cm², 46 Joules/cm², 47 Joules/cm², 48 Joules/cm², 49 Joules/cm², or 50 Joules/cm². Light can be administered over time to confer the appropriate dosage. For example, the total dosage can be delivered in a period from 1 second to about 15 minutes (e.g., 1, 2, 3, 4, 5, 6, 7, 8, 9, 10, 11, 12, 13, 14, or 15 minutes). In an embodiment, 6 Joules/cm² per minute is delivered over a 5 minute time period for a total dosage of 30 Joules/cm².

The optic fiber used in the present invention may have a diameter ranging from about 100 μm to about 500 μm (e.g., 150 μm, 200 μm, 250 μm, 300 μm, 350 μm, 400 μm, or 450 μm), and a length ranging from about 5 mm to about 40 mm (e.g., about 10 mm, 15 mm, 20 mm, 25 mm, 30 mm or 35 mm)

### Power Source:

FIG. 1 also shows battery 8 which is coupled to light source 18 and the circuit board 16. Battery 8 resides in battery compartment 10. Any self-contained power source can be used with the present invention. A power source is a source of electricity used to provide power to one or more light sources. One or more power sources can be used and they can be rechargeable or non-rechargeable. Rechargeable refers to a battery whose electrical energy can be restored, either fully or partially, to a charged state by passing an electrical current in the opposite direction through the cell. In the case of a rechargeable battery, when the device is not in use, the battery of the device can be put into communication with an electrical current, in the proper direction to be recharged. This can be accomplished by using a base, plug, or adaptor that is attached to the device and then plugged into an electrical outlet. The device can be adapted to receive the base, plug, or adaptor. For example, the device can include contacts or prongs which, when set into a base, receives an electrical current that recharges the battery. Alternatively, the device can have an opening to receive a plug that provides an electrical current. Yet another embodiment includes a removable battery that can either be replaced and/or recharged in a battery charging device. Methods, devices and adaptations for recharging batteries are known in the art and can be used with the present invention to recharge one or more batteries used in the device.

A “self contained power source” refers to a power source that has sufficient power to run the device during use (e.g., for at least a single or routine use) without the need of an external power source. Self contained power sources can be recharged when not in use. Examples of batteries that can be used by the present invention include those used for watches and hearing aids (e.g., Zinc Silver Oxide, Lithium ion). Other examples of specific types of batteries include Alkaline batteries, Nickel-iron, Lead, Gel, Absorbed glass mat, Nickel-cadmium, Nickel metal hydride, Lithium ion polymer, Sodium-sulfur, Nickel-zinc, Molten salt, Super iron, and Zinc-bromine flow. The battery can have any shape that fits into the device having a pocket, housing or area which can accommodate the size of the battery.

### Oxygen Supply:

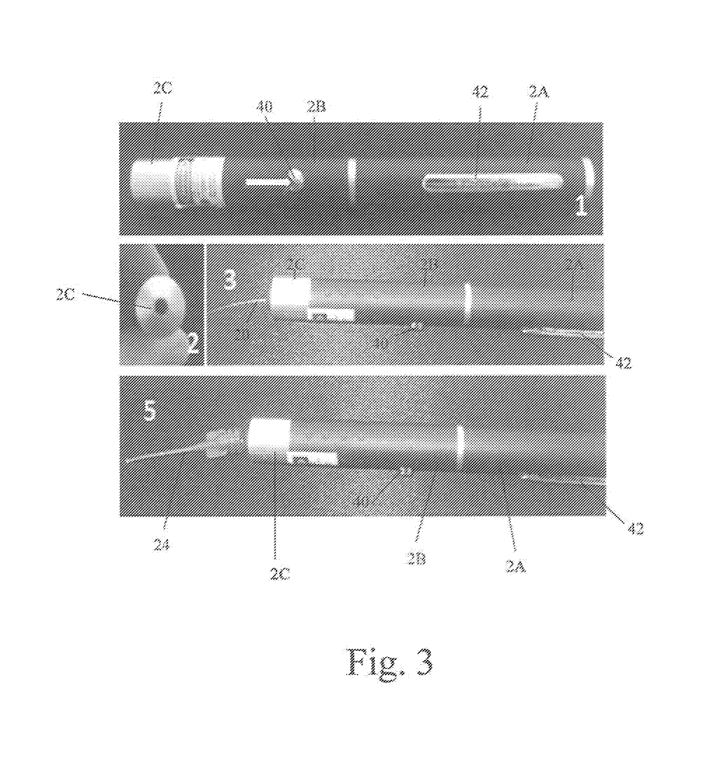

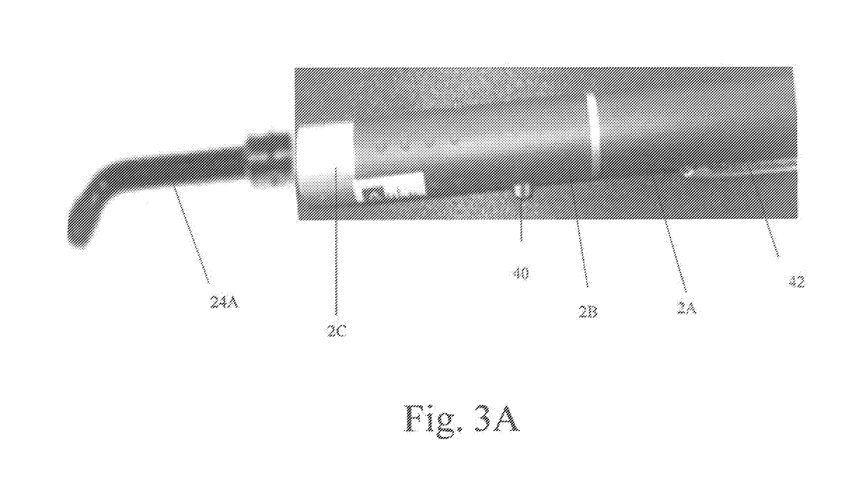

The handheld dental device 100 also has oxygen tank 4 which contains a supply of compressed oxygen. The oxygen tank 4 of this embodiment is pierced by pin 12 at oxygen neck 6. Pin 12 extends from the walls of middle housing 2B. When the pin pierces the oxygen tank, oxygen flows from the tank through passage 14, which is defined by the walls of the middle housing and the compartments that hold the battery and circuit board. The passage continues to applicator tip 24 and oxygen is delivered along with light. When the user engages switch 40 (see FIGS. 3 and 3A), oxygen is released and flows through the passage and out from the tip.

The oxygen tank can be a cylinder made to hold high pressured gasses. Oxygen tanks are made generally from aluminum, cast in a cylindrical shape, finished and filled with liquid air to created pressurized oxygen. Oxygen tanks can hold between about 500 and about 2,000 lb/in². The oxygen tank for use with the present invention may hold between about 1 ml to about 200 ml of pressurized oxygen (e.g., about 50, 100, 150 ml of pressurized oxygen). The amount of oxygen in the tank is suitable for at least one treatment of photodynamic therapy to the targeted area. The device, for example, can provide a cloud or puff of oxygen which is enough to provide photodynamic therapy. In an embodiment, the tank can hold enough oxygen for multiple treatments.

The oxygen tank may be replaceable with filled oxygen tanks. For handheld device 100, back housing 2A is unscrewed from middle housing 2B, and tank 4 is removed. It is replaced with a filled tank and the back housing is screwed onto the middle housing. When connecting the housing compartments, pin 12 pierces the seal on oxygen tank 4 to allow flow of oxygen within passage 14.

In another embodiment, a continuous supply of oxygen can be provided. For example, an external oxygen supply can be present e.g., in a dental office and connected to the handheld device to provide the supply of oxygen to the applicator tip. Accordingly, a means for supplying oxygen can be in the form of an oxygen tank, external or internally housed, or a continuous supply of concentrated oxygen. Additionally, the oxygen tank of the device of the present invention may be refillable using the oxygen supply present in a dental office. In such a case, a means to refill the oxygen tank can be provided as an option.

### Interchangeable Applicator Tip:

Applicator tip 24 is shown in FIGS. 1, 3 and 3A. Fiber 20 is inserted into applicator tip 24. In certain embodiments, the applicator tip is sterile and disposable. In an embodiment, the applicator tip has a plurality of pores, such as pores 26A-D, to allow for the delivery of oxygen from the passage to the tissue to be treated. In an embodiment, the applicator tip is conical in shape and has a length of between 5 mm and 20 mm (e.g., between 10 mm and 14 mm). Oxygen may be delivered through the tip to the targeted tissue at a rate of between about 1 ml/min and about 10 ml/min (e.g., between about 1 and about 5 ml/min). In an embodiment, administration of 5 min of light with an oxygen tank containing 100 ml of pressurized oxygen, the flow rate will be between about 1 and about 5 ml/min.

The applicator tip can be made from a plastic material that can withstand the heat of the light. The applicator tip, in one embodiment, is flexible and allows the tip to conform to the shape of the target area, such as a root canal. In an embodiment, the applicator tip, having a lumen for receiving the optic fiber, can bend along with the fiber during use, as described herein.

The applicator tip may be made from material that allows the light from the optic fiber to pass through with minimal refraction of the light. In some embodiments, the applicator tip may be made from material that enhances the light as it passes through it. In an embodiment, the light is enhanced at the tip because the infection is stronger or more pervasive at the lower third of the tooth.

Additionally, as shown in FIG. 3A, applicator tip 24A is interchangeable to provider a larger diameter for emission of light to be utilized in the pulpal chamber or the surgical site (bony crypt) or after restorative preparation for utilization in vital pulp therapy (including pulpal regenerative procedures), disinfection of the surgical site (apical root preparations in endodontic periradicular surgery) and disinfection of the prepared clinical crown before insertion of direct/indirect restorations, respectively. The present invention can be used for a flapless endodontic surgical procedure and insertion of a rigid needle-like apparatus directed toward apical tissue.

### Switch, Timer and Circuit Board

In some embodiments, the device includes a switch, timer, or circuit board.

The handheld photodynamic therapy device of present invention, in an aspect, can also include switch 40. See FIG. 3. The switch can control release of the light and the oxygen supply so that the targeted area can be treated. Accordingly, the switch can communicate with circuit board 16 to turn on the LED so that the LED emits light that travels down the optic fiber. Additionally, the switch can communicate with a door or barrier that controls the release of the oxygen from the passage to the applicator tip, which in turn, flows through the pores of the tip to the target area to be treated. The switch can be coupled with a timer, controlled by the circuit board so that the administration of the light and oxygen can be controlled.

The device can optionally further include a timer that causes the light to turn off once a certain time is reached. For example, the timer can be set to allow the light, oxygen or both to be administered for a time period between 1 second to 15 minutes (e.g., 1 minute, 2 minutes, 3 minutes, 4 minutes, 5 minutes, 6 minutes, 7 minutes, 8 minutes or 9 minutes, 10 minutes, 11 minutes, 12, minutes, 13 minutes or 14 minutes). In an embodiment, the time period for light administration is about 5 minutes. The light and oxygen can be administered simultaneously or sequentially so long as the desired effect of reducing and/or inhibiting bacteria is achieved. One or more timers can be used for light, oxygen or both. The timer can be used to time each light/oxygen session, and/or measure time between sessions. As such, timers can be used to control the amount of light/oxygen therapy delivered and to avoid overexposure. Timers can be mechanical, electrical, or digital timers or any combination thereof, and integrated into the device. An example of an electrical timer that can be used with the present invention includes a thermal expansion timer. Using a thermal expansion timer, electrical current passes through a finger made of two types of metal. Heat causes the finger to expand on one side faster than the other, and as the finger moves away from a contact, the device can turn off. Any timer known in the art or developed in the future can be used with the present invention.

Additional time can be allotted for the application of the photosensitizer. A photosensitizer can be applied for a period of time prior to the light administration. For example, a photosensitizer can be applied for a period of time between 1 second to 15 minutes (e.g., 1 minute, 2 minutes, 3 minutes, 4 minutes, 5 minutes, 6 minutes, 7 minutes, 8 minutes or 9 minutes, 10 minutes, 11 minutes, 12, minutes, 13 minutes or 14 minutes). In an embodiment, the time period for allowing the photosensitizer to be in contact with the tissue is about 5 minutes. In such an embodiment, the photosensitizer can be applied for a period of 5 minutes and light administration can be applied for 5 minutes for a total treatment time of 10 minutes.

The amount of time for administration of both oxygen and light can depend on the target area being treated and the condition being treated. Additionally, in an embodiment, the light and oxygen are administered simultaneously. However, they can also be administered sequentially in time or in an overlapping method so long as the desired effect of the photodynamic therapy is achieved (e.g., reducing the number of bacteria at the targeted site).

Switch 40 is electrically connected to battery 8, light emitting diode 18, circuit board 16 and/or a door/barrier for controlling oxygen flow. In an embodiment, the switch is an open switch and has two contacts. The switch remains open until the contacts are connected. When the user depresses the switch, the electrical circuit is completed. The contact material can be any conductive material known in the art, or developed in the future. The number of switches and/or contacts can vary and can be arranged in any manner to allow a completion of the electrical circuit. Switches can be used together with other settings or controls to direct which light sources and oxygen flow. Various arrangements with settings, controls and switches can be used to accommodate a variety of uses that include caries treatment, root canal treatment, gum treatment, and other uses described further herein. Settings can be used to vary the output of light and oxygen delivered.

Any type of switch can be used to complete the circuit so long as the switch can be activated by the user. Types of switches include those known in the art and those developed in the future. More than one switch and/or arrangement of switches can be used. For example, a switch can be used to engage the light source and another to engage the oxygen flow.

The material used for the contacts of the switch, and for providing electrical communication (e.g., wires, bands, plates) between the parts of the device, can be chosen based on the material's properties including its ability to resist corrosion, electrical conductivity, mechanical strength, cost and toxicity, and the like. Switches, the power source and the light sources can be used in conjunction with other electrical components including resistors, capacitors, circuit boards, heat sinks, light distributors, light pipes, insulators, and others known in the art.

### Administration

In some embodiments, the present invention relates to methods of administering light and oxygen to the oral cavity of an individual using a device described herein. The administration of a photosensitizer can be applied by the user manually or using the device (e.g., in the case the device has a chamber for storing and administering the photosensitizer). The administration of light, oxygen and/or the photosensitizer, as demonstrated in the Exemplification, can be used to reduce the overall number of one or more bacteria and/or fungi in the oral cavity. In addition to reducing the overall bacteria/fungi count, the methods of the present invention may embody treating root canal infections; treating caries, peri-implantitis, and oral candidiasis, reducing halitosis, improving a gum condition (e.g., gingivitis or periodontitis), and improving overall oral health or any combination thereof. The methods involve applying or administering light, oxygen and/or the photosensitizer using the device of the present invention, as described herein.

The term, “administering,” refers to providing an amount of light, oxygen and/or a photosensitizer to a portion of the oral cavity. Providing light, oxygen and optionally a photosensitizer is referred to herein as “photodynamic therapy.” An “effective amount” or “therapeutically effective amount” may refer to the amount of light, and oxygen and, optionally, a photosensitizer which reduces, in whole or in part, the growth or proliferation of one or more bacteria associated with an oral disease or condition. In particular, the amount of light, oxygen and/or photosensitizer administered is one that treats or ameliorates one or more oral health symptoms, including, but not limited to providing, an anti-bacterial effect, a sterilizing effect, an endodontic improvement effect, or a periodontal improvement effect. The therapeutically effective amount of light, oxygen and/or a photosensitizer can be used for prevention and/or treatment purposes. In an embodiment, the therapeutically effective amount of light can be at the predetermined wavelength at predetermined light dosages, as described herein.

“Treating” refers to improving or making better one or more symptoms associated with the disease or condition, as compared to the state of the symptom prior to treatment, or as compared to a control. “Improving oral health” refers to ameliorating one or more symptoms associated with oral diseases or conditions. Examples of such symptoms include bad breath, increased overall count of one or more bacteria, gum bleeding, gum swelling, inflammation, lesions (e.g., cancerous and pre-cancerous), pain, decay, sensitivity, and slow healing. Symptoms of oral diseases or conditions are known in the art and recognized by a dentist.

Regardless if a timer, as described herein, is used, the duration of exposure of the light and oxygen to the teeth and/or gums can range from about 1 second to about an hour, or about 5 seconds to about 15 minutes, or about 5 seconds to about 5 minutes, or about 5 seconds to about 2 minutes, or from about 5 seconds to 1 minute. The duration of exposure can be specifically 5 seconds, 10 seconds, 15 seconds, 30 seconds, 45 seconds, 1 minute, 2 minutes, 3 minutes, 4 minutes, 5 minutes, 10 minutes, 15 minutes, 20 minutes, 30 minutes, 40 minutes, 50 minutes, or one hour. In some embodiments, the light source can automatically turn off after the duration of application. As higher light intensity is reached, the duration of exposure can decrease.

The frequency of application of light to the oral cavity can be on a periodic basis (e.g., a few times per visit) at one or more visits per year. The application of light can be intermittent, pulsed, or continuous with each application. Light can be administered continuously, intermittently, or combinations thereof, over a period of time.

In particular, one or more of the following variables relating to the light exposure may be encompassed by the present invention, (1) the type of light source used; (2) the intensity/irradiance of the light; (3) the wavelength of the light emitted from the light source; (4) the duration of the exposure of the light to the teeth and gums; and (5) the frequency of application. The variables, in certain aspects, depend on another. For example, an increase in the duration of exposure can decrease the intensity of the light emitted.

As described herein, methods of the present invention may relate to reducing the number of one or more bacteria in the oral cavity by providing or administering light using the device described herein. This may be performed, in one aspect, by administering light at the wavelength and power, also as described herein. An agent that assists in eliminating bacteria can be also administered before, during or after treatment. Examples of such agents are chlorine dioxide, chlorhexidine, triclosan, chelators (ethylenediaminetetracetic acid), cleaning agents, and oxidizing agents. Other such agents include: 1) a mixture of doxycycline, citric acid, and a detergent (Tween 80)—“MTAD”; or 2) a mixture of dimensionally stable ethylenediaminetetracetic acid and chlorhexidine, referred to as “QMix”).

### Channel/Compartment for Photosensitizer

The device can further include one or more compartments, channels or storage areas for a photosensitizing agent, or a solution or gel that aids in the use of the device. Such storage areas can be used to store solutions that assist light/oxygen in killing or reducing the number of bacteria. Such storage areas can be refillable or replaced with cartridges. Such storage areas or channels, in an embodiment, are positioned alongside the oxygen passage, or elongated wall of the device, or elsewhere in the device. The user can apply the solution prior to or during administration of the light and/or oxygen. In another embodiment, the solution or gel can be released periodically or continuously during use of the device e.g., using a timer. Such solutions or gels can be placed on the targeted area by the user prior to, during or after use of the device of the present invention. Types of solutions that can be used with the device include solutions that kill bacteria and examples are chlorine dioxide, chlorhexidine, triclosin. Other solutions that kill bacteria include photosensitizers (e.g., methylene blue, acridine orange, porfimer sodium, benzoporphyrin derivative, methoxsalen, psoralen, talaporfin, temoporfin, verteporfin and the like).

Certain agents that may be used in the methods of the present invention are agents that improve oral health by enhancing or assisting in the effect of light/oxygen therapy administered as described herein. Agents are referred to herein as a compound that enhances or increases the effect of the light therapy. Agents can be administered as gels, solutions, or in strips. Any combination of agents along with the device can be used to carry out the methods of some embodiments of the present invention. For example, a photosensitizer or oxidizing agent can increase the number of bacteria being killed when used with the light/oxygen device of the present invention. Additionally, antimicrobial chemical irrigants can be used to further reduce the overall bacteria number.

The agents are administered by applying the agent to one or more portions of the oral cavity. The actual effective amounts of an agent can vary according to the specific agent being utilized, the particular composition formulated, and the condition of the patient, for example. As used herein, an effective amount of an agent is an amount that can reduce or make better one or more symptoms associated with an oral disease or condition. Dosages for agents are known in the art or can be determined using methods and protocols known in the art, such as conventional pharmacological protocols. Light and oxygen in combination with one or more agents can be administered. When administering light, oxygen and one or more agents, the administration can occur simultaneously or sequentially in time. The agent, oxygen, and/or light can be administered before and after one another, or at the same time. Thus, the term “co-administration” is used herein to mean that the agent, oxygen and/or light will be administered at times to reduce the number of bacteria, improve oral health or reduce the occurrence, severity of one or more symptoms associated with oral disease or condition, as described herein. The methods of the present invention are not limited to the sequence in which the light, oxygen and/or one or more agents are administered; so long as they are administered close enough in time to produce the desired effect. The methods also include co-administration with other drugs that are used to treat oral diseases or conditions.

### Methods of Treatment & Microbiology and Pathogenesis of the Root Canal Infections:

Bacterial infection of the dental pulp is responsible for the consequent soft tissue necrosis, and destruction of periapical bone. The predominant taxa include: Prevotella, Porphyromonas, Fusobacterium, Peptostreptococcus, Eubacterium, anaerobic Streptococci, Treponema, etc. Acute symptoms (pain, swelling) have been associated with infection by black-pigmented Porphyromonas and Prevotella species. 16S rRNA sequencing demonstrates a much greater diversity of infecting species. Thus, 65 taxa, including 27 novel taxa and 26 non-cultivatible taxa were identified in only 5 root canal samples.

Generally, endodontic failures are caused by the proliferation of residual bacteria within the root canal system. Studies have convincingly demonstrated that endodontic success rates are substantially higher for teeth with vital pulps, and hence not grossly infected at the time of treatment, compared to grossly infected teeth with necrotic pulps. Similarly, teeth without bacterial growth at the time of root canal filling have a higher success rate than teeth that do. Although the bulk of the infecting microorganisms are removed during endodontic instrumentation, residual bacteria are readily detectable in approximately one-half of teeth at the time of placement of a filling material, despite extensive irrigation with sodium hypochlorite. The actual proportion of infected teeth is probably even higher, if PCR based methods for detection of bacteria were to be utilized. The device of some embodiments of present invention is able to further reduce the number of bacteria to reduce or prevent endeondotic failure. The presence of uninstrumented areas of dentin with grooves and pulpal remnants cannot be disinfected by irrigants. Thus infected pulpal remnants harbor residual microorganisms which can reinfect the root canal space. Similarly dentinal tubules present a honeycomb structure that allows persistent infection caused by anaerobic bacteria. Scanning electron microscopic investigations have demonstrated bacterial penetration from 150 to up more than 700 μm into dentinal tubules. The presence of a ‘smear layer’ after instrumentation of teeth reduces the effectiveness of irrigants and temporary dressings in disinfecting dentinal tubules. Data such as these strongly indicate that the outcome of endodontic treatment is highly dependent upon the completeness of removal or killing of bacteria in the root canal system. The present invention may allow for the removal or killing of bacteria in the root canal system.

Clinical signs of endodontic failure include: the persistence or exacerbation of pain to percussion/palpation consequent to root canal therapy; presence of a draining sinus tract; development of a periapical radiolucency (where none existed previously), or expansion of a pre-existing periapical radiolucency. Residual bacteria stimulate a pro-inflammatory and immune response in the periapical tissues, that results in osteoclast recruitment, activation and bone resorption.

A number of studies have examined the microorganisms that are present in root canals in cases of treatment failures. These studies have mostly employed anaerobic culture techniques, and hence are able to identify obligate anaerobes as well as faculatative organisms. However, given that as many as 50% of oral microorganisms are cultivable even using state-of-the-art techniques, these studies are somewhat limited in that they assess only a proportion of the microorganisms that may be present.

Despite this proviso, some important data have been generated concerning the flora associated with endodontic failures. Two other, more recent studies have utilized molecular methods based on PCR amplification of the microbial 16S rRNA genes. These studies can detect both cultivable and non-cultivable organisms, although they are largely non-quantitative given the often extreme degree of amplification of the target sequences.

The details of these studies are summarized in Table III (from Hancock et al, Oral Surgery Oral Medicine Oral Pathology Oral Radiology Endodontics; 91(5):579-86 (2001)), and Table V (from Siqueira & Rocas, Oral Surg Oral Med Oral Pathol Oral Radiol Endod. January; 97(1):85-94 (2004)).

TABLE III
Comparison with Scandinavian studies

| | Meller | Molander | Sundqvist | Hancock |
|---|---|---|---|---|
| Finding | (1966) | et al (1998) | et al (1998) | et al (2000) |
| Bacterial species per | 1.6 | 1.7 | 1.3 | 1.7 |
| root canal with | | | | |
| bacteria | | | | |
| Anaerobic bacteria* | 51 | 39 | 42 | 42 |
| Gram-positive | 80 | ND | 87 | 80 |
| bacteria* | | | | |
| E. faecalis† | 29 | 47 | 38 | 32 |

When compared with previous studies that were carried out in Scandinavia, there was no statistically difference in any of the measures of bacteria grown. including the percentage of canals that were positive for E. faecalis.
ND, Value not given.
*Percent of isolated bacteria.
†Percent of canals with bacteria.

TABLE V
Comparison of findings of studies on the microorganisms
associated with root-filled teeth with periradicular lesions

| | | Samples positive for | | |
|---|---|---|---|---|
| | Identification | the presence of | Enterococcus | Candida |
| Study | methods | microorganisms | faecalis | albicans |
| Meller³² (1966) | Culture | 57% | 29% | 3% |
| Sundqvist et al¹⁰ (1998) | Culture | 44% | 38% | 8% |
| Molander et al⁹ (1998) | Culture | 68% | 47% | 4% |
| Peciuliene et al³⁴ | Culture | 80% | 70% | — |
| (2000) | | | | |
| Peciuliene et al³⁵ | Culture | 83% | 64% | 18% |
| (2001) | | | | |
| Hancock et al¹¹ (2001) | Culture | 63% | 30% | 3% |
| Cheung and Ho³⁷ | Culture | 67% | 0% | 17% |
| (2001) | | | | |
| Rolph et al¹⁶ (2001) | PCR | 91% | 0% | — |
| Pinheiro et al³⁶ (2003) | Culture | 85% | 53% | 4% |
| This study | PCR | 100% | 77% | 9% |

PCR, Polyincrease chain reaction.

In the aggregate, these studies illustrate several key points. (1) Microorganisms can be detected in the majority of failing cases using cultural methods, and can be identified in virtually all cases using high sensitivity PCR-based approaches. (2) The data indicate that the bacteria present in endodontic failures are distinct from those present in infected root canals prior to endodontic treatment. Thus, failing cases are associated with high proportions of gram positive aerobic and facultative anaerobic organisms, vs the predominance of strict anaerobes at presentation. Enterococcus faecalis, which is rarely found in large proportions in untreated root canals, is highly associated with failures, as are Candida albicans and Actinomyces sp. (3) On the other hand, it should be noted that some studies have implicated other taxa including Pseudomonas, Staphylococci and Streptococci as causative of failures. Certain studies report large numbers of isolates including Actinomyces, Streptococci, Peptostreptococcus and Prevotella in addition to Enterococci. 16S rRNA analyses have given an even more diverse picture of bacteria associated with failure (Id.), with species including Pseudoramibacter, Proprionibacterium, Dialister, and Filifactor prominent in addition to Enterococcus. Thus, although Enterococcus has received much attention, it is clear that other species are implicated in causing endodontic treatment failures.

Taken together, these data indicate that the subset of gram positives is more resistant to instrumentation and irrigation procedures than are the obligate anaerobes, and that they are essentially selected for by treatment procedures. In cases in which the root canal filling is inadequate (e.g., lacks an apical seal and contains voids into which tissue fluids can percolate), these residual organisms are most likely to proliferate, providing a nutrient source and leading to microbial proliferation. This scenario has been substantiated in controlled studies that examined changes in residual root canal bacteria after treatment; facultative bacteria were more resistant to treatment than anaerobes in a monkey model.

Accordingly, an embodiment of the present invention provides for a method of reducing bacteria that is not or cannot be reduced by traditional dental procedures.

### Reducing Bacteria and Fungi

The present invention relates to methods for reducing and/or eliminating one or more bacteria and/or fungi. Such bacteria include bacteria of the taxa: Prevotella, Porphyromonas, Fusobacterium, Peptostreptococcus, Eubacterium, anaerobic Streptococci, Treponema, Candida albicans, Psuedomonas, Staphylococci, Actinomyces, Streptococci, Enterococci, Pseudoramibacter, Proprionibacterium, Dialister, and Filifactor. In particular, the following endodontic pathogens were studied, as described in the Exemplification, and found to be reduced with photodynamic therapy: Porphyromonas gingivalis (ATCC 33277), Prevotella intermedia (ATCC 25611), Fusobacterium nucleatum nucleatum (ATCC 25586), Peptostreptocococcus micros (ATCC 33270), Porphyromonas endodontalis (ATCC 35406), and Enterococcus faecalis (ATCC 29212). Examples of fungi include Candida albicans, Candida glabrata or Candida tropicalis.

To determine if the number of bacteria and/or fungi is reduced, a sample from the oral cavity can be obtained. Bacteria can be grown, cultured or tested to determine which bacteria exists and their overall number. Alternatively, the nucleic acid of the bacteria of the sample can be assayed using gene chip technology or nucleic acid hybridization techniques. Methods of determining the types and number of bacteria and/or fungi that exist in a sample are known in the art. After photodynamic therapy administration occurs using one embodiment of the present invention, another sample can be obtained. The results from the two samples are compared, and a reduction of the number of one or more bacteria and/or fungi occurs. In another embodiment, bacteria and/or fungi levels after treatment can be compared to a control (e.g., the average levels of bacteria in a similar patient population or pool) to determine that bacteria and/or fungi levels have been reduced. In an embodiment, the methods described herein reduces or eliminates from about 5% to about 25%, about 5% to about 50%, about 5% to about 75%, or about 5% to about 100% of one or more bacteria and/or fungi present in the oral cavity. In another embodiment, from about 5% to about 25%, about 5% to about 50%, about 5% to about 75%, or about 5% to about 100% of black-pigmented bacteria in the oral cavity is reduced after exposure to light. The reduction of certain bacteria and/or fungi also improves the overall oral health. Similarly, a reduction in the number of bacteria and/or fungi prevents or treats symptoms of systemic disease associated with oral conditions such as heart disease, diabetes, low birth-weight babies and oral candidiasis.

### Endodontic Therapy of Root Canals

The methods of the present invention may include treating root canals infected with bacteria, as described herein. Hence, the present invention may include administration of photodynamic therapy to reduce the number of one or more bacteria infecting the root canal. Treatment of an infected root canal involves the mechanical and chemical removal of nerve tissue, blood vessels and pulpal remnants and disinfection of the canal(s), with hand files and engine driven rotary files and irrigating solutions. The disinfected root canal space is then filled with an inert filling such as gutta percha and typically an eugenol-based cement. Once the nerve and infected pulpa; tissue of the tooth may be removed, using the device of the present invention, photodynamic therapy can be applied to the root canal to reduce the number of residual bacteria in the canal that is not accessible with current mechanical and chemical clinical protocols. This can be applied before or after, or in place of, the disinfection of the canal with irrigating solutions. Application of photodynamic therapy using the device of the present invention reduces the number of bacteria in the root canal and reduces the risk that the endodontic therapy fails. Specifically, the present invention may increase the success of clinical outcomes by reducing failure of endodontic therapy by at least 5% to about 100% (e.g., by at least about 10%, 20%, 30%, 40%, 50%, 60%, 70%, 80%, 90% or by 100%), as compared to the same endodontic therapy without the application of light, oxygen and/or a photosensitizer.

### Periodontal (Gum) Conditions

Similarly, methods of the present invention may include improving a user's periodontal condition (e.g., gingivitis, peri-implantitis, or periodontitis). Accordingly, the present invention may include administration of photodynamic therapy to improve one or more symptoms associated with the periodontal conditions. For example, gingivitis is a soft tissue gum condition whose symptoms include the bleeding of gums and gum swelling. After photodynamic therapy administration, as described herein, one or more of these symptoms are decreased. Similarly, symptoms of periodontitis, in addition to periodontal bleeding and gum swelling, include the formation of perio-pockets, pockets between the gum and teeth. As such, photodynamic therapy administration, in an embodiment, decreases the size of these pockets, along with decreasing gum bleeding and/or gum swelling.

### Prophylactic Treatment/Cleaning

In yet another embodiment, the device of the present invention for administering photodynamic therapy can be used during dental cleanings. Dental cleanings involve mechanical removal of plaque and prophylactic application of topical gels such as fluoride treatment. Photodynamic therapy using the device of the present invention can also be included as a step during the dental cleaning procedures. Such therapy can be used to reduce halitosis. Photodynamic therapy can be used as a method of reducing bacteria that causes caries so as to reduce the prevalence, incidence and/or severity of caries in the oral cavity. In an embodiment, the application of photodynamic therapy using the device of the present invention reduces the incidence and/or severity of caries in a range between about 5% and 100% (e.g., by at least about 10%, 20%, 30%, 40%, 50%, 60%, 70%, 80%, 90% or by 100%), as compared to using the same dental cleaning procedure but without the application of light, oxygen and/or a photosensitive

### Kits

The present invention may further include systems and kits for improving oral health. Such systems and kits include the device described herein, and an item or agent associated with improving oral health or using the device. Agents include those that enhance the effect of the light therapy administered by the device, as described herein. Such items associated with using the device include batteries and/or a device, plug or adapted to charge the battery.

This above description and following examples are not to be taken in a limiting sense, but are made merely for the purpose of illustrating the general principles of the invention. The section titles and overall organization of the present detailed description are for the purpose of convenience only and are not intended to limit the present invention.

### EXEMPLIFICATION

The work that led to the actual device proposed herein was built on the following interdependent foundations:

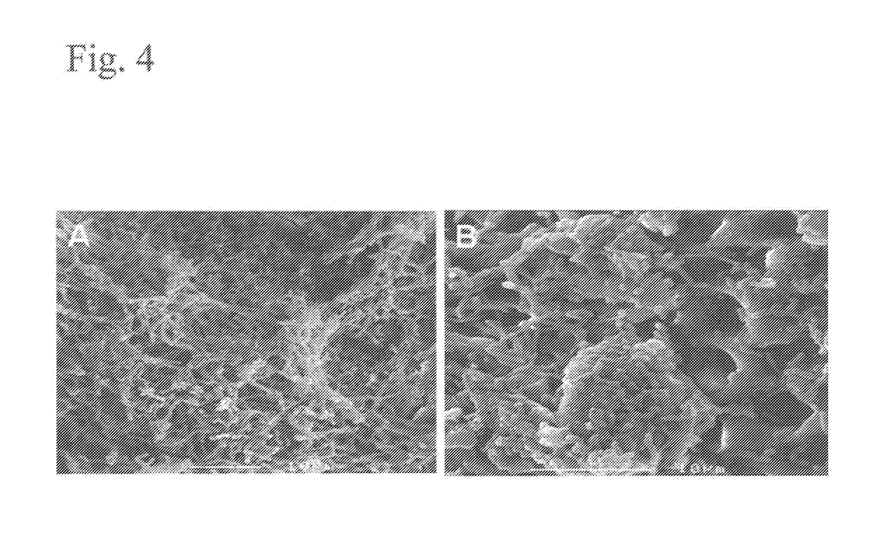

1) The development and characterization of mono- and multi-species biofilm models in experimentally infected root canals of extracted teeth (FIG. 4);
2) The utilization of an FDA approved drug—methylene blue (MB)—as the photosensitizer;
3) The investigation of the photodestruction of root canal biofilms in vitro;
4) The development of a novel light delivery system that maximizes the distribution of light within the entire anatomy of the root canal system rather than unidirectionally (FIG. 2);
5) The ongoing refinement of light and drug dosimetry;
6) The assessment of the safety of photodynamic therapy (PDT); and
7) The evaluation of the anti-microbial effects of PDT in a naturally-infected model (infected human teeth ex vivo).

### Example 1

Development and characterization of mono- and multi-species biofilms in experimentally infected root canals of extracted teeth and their destruction by PDT.

A novel fiber optic delivery system was developed that efficiently delivers 665 nm light at 360° to the entire root canal system (FIG. 2). Drug and light parameters needed to achieve elimination of root canal microbial biofilms were investigated. (Soukos N S et al (2006). Photodynamic therapy for endodontic disinfection. J Endod, 32 (10): 979-984.)

The effects of PDT were investigated on endodontic pathogens in planktonic phase as well as on Enterococcus faecalis biofilms in experimentally infected root canals of extracted teeth. Strains of Porphyromonas gingivalis (ATCC 33277), P. intermedia (ATCC 25611), Fusobacterium nucleatum nucleatum (ATCC 25586), Peptostreptocococcus micros (ATCC 33270), Porphyromonas endodontalis (ATCC 35406), and E. faecalis (ATCC 29212) were sensitized with methylene blue (MB) (25 μg/ml) for 5 minutes followed by exposure to res light of 665 nm with an energy fluence of 30 J/cm². MB fully eliminated all bacterial species with the exception of E. faecalis (53% killing) (Table 1). In Table 1, Surviving species are expressed as percentage-of-controls receiving neither methylene blue nor light. Each value is the mean of values obtained from 2 to 4 experiments (±standard error). The same concentration of MB in combination with red light (222 J/cm²) was able to eliminate 97% of E. faecalis biofilm bacteria in root canals. Light was uniformly delivered at 360 degrees in the root canal system with a flexible optical fiber with cylindrical diffusers and a diameter of 500 μm. Table 1 presents the survival of endodontic bacteria after their incubation only with methylene blue (25 μg/ml) or methylene blue (25 μg/ml) and red light (30 J/cm²):

TABLE 1

| Species | Only methylene blue | PDT |
|---|---|---|
| P. micros | 21 ± 10.6 | 0 |
| E. faecalis | 94.5 ± 17.6 | 47 ± 12.1 |
| F. nuc. nuc. | 5.5 ± 0.4 | 0 |
| P. intermedia | 0 | 0 |
| P. gingivalis | 6.7 ± 0.1 | 0 |
| P. endodontalis | 0 | 0 |

Foschi F et al (2007). Photodynamic inactivation of Enterococcus faecalis in dental root canals in vitro. Lasers Surg Med, 39 (10): 782-787.

The photodynamic effects of methylene blue (MB) on Enterococcus faecalis species in experimentally infected root canals of extracted teeth after their sensitization with a concentration of MB that exhibits reduced dark toxicity were investigated. In a model of root canal infection, 64 root canal specimens were prepared from extracted, single-rooted teeth and inoculated with E. faecalis (ATCC 29212). Three days later root canal infection was confirmed by scanning electron microscopy. The root canal systems were then incubated with 6.25 μg/ml MB for 5 minutes followed by exposure to light at 665 nm (60 J/cm²) that was delivered from a diode laser via a fiber optic with a diameter of 500 μm. Following PDT the canal content was sampled by flushing the root canals, serially diluted and cultured on blood agar. Survival fractions were calculated by counting colony-forming units. PDT achieved 77.5% reduction of E. faecalis viability. MB alone and light alone reduced bacterial viability by 19.5% and 40.5%, respectively. (Fimple J L et al (2008). Photodynamic treatment of endodontic polymicrobial infection in vitro. J Endod, 34 (6): 728-734). The photodynamic effects of methylene blue (MB) on multispecies root canal biofilms comprising Actinomyces israelii, Fusobacterium nucleatum subspecies nucleatum, Porphyromonas gingivalis, and Prevotella intermedia in experimentally infected root canals of extracted human teeth were investigated in vitro. The 4 test microorganisms were detected in root canals by using DNA probes.

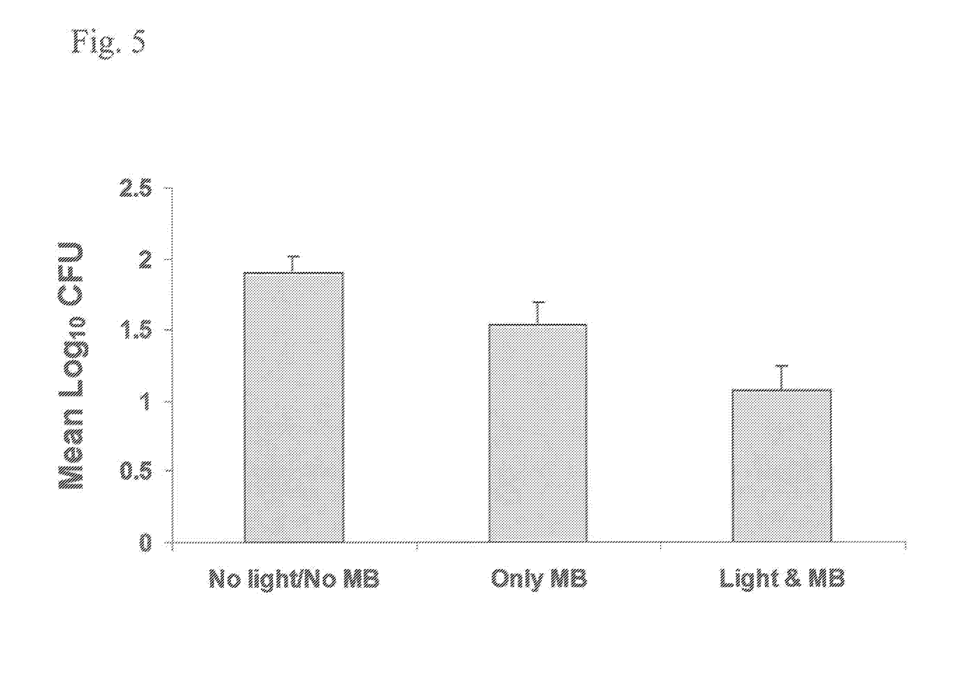

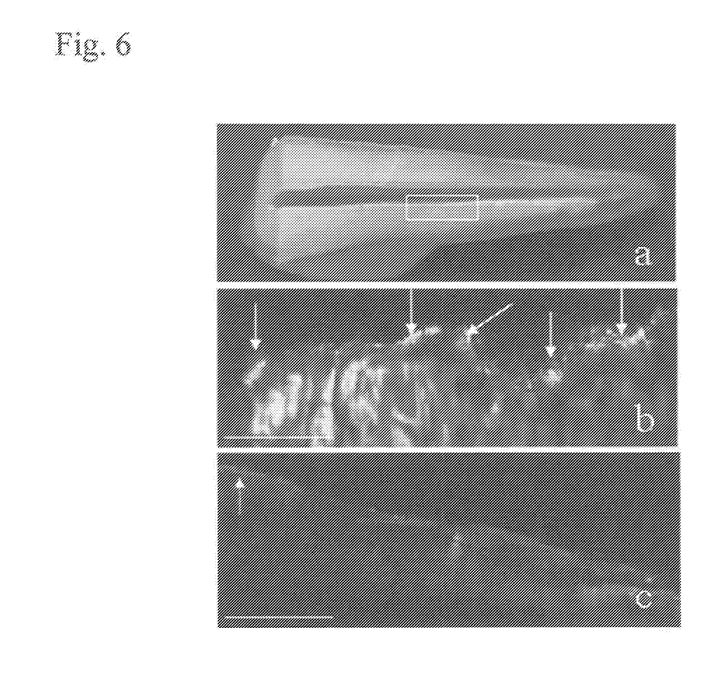

Scanning electron microscopy showed the presence of biofilms in root canals before therapy (FIG. 4). Root canal systems were incubated with MB (25 μg/mL) for 10 minutes followed by exposure to red light at 665 nm with an energy fluence of 30 J/cm². Light was delivered from a diode laser via a 250-μm diameter polymethyl methacrylate optical fiber that uniformly distributed light over 360 degrees (FIG. 2). PDT achieved up to 80% reduction of colony-forming unit counts (CFU) (FIG. 5). In FIG. 5, each bar is the mean Logic, CFU levels (+standard error). The combination of light and MB was significantly lower than either control or MB alone (B). Confocal Scanning Laser Microscopy images were obtained from the mid-root area of canals (FIG. 6A, box). FIG. 6B is a X-Z confocal image that shows the presence of microbial biofilms (arrows) in the root canal system (green areas represent viable microbial masses that extend into the dentinal tubules), with a thickness of 20-25 μm. Another X-Z confocal image (FIG. 6C) demonstrates the destruction of biofilm species by photodynamic therapy (red areas represent dead species). A few foci of residual live microorganisms (arrow) were also detected.

### Example 2

### PDT Safety Assessment

There is a safe therapeutic window whereby microorganisms can be inactivated without affecting host cell viability, and PDT does not induce apoptosis of mammalian cells. (Xu Y et al (2009). Endodontic antimicrobial photodynamic therapy: Safety assessment in mammalian cell cultures. J. Endod. 35(11):1567-1572). When a photoactive compound is applied in the root canal system, it is taken up by residual bacteria in the main canals, isthmuses, lateral canals, and dentinal tubules. It is also possible that this compound may escape into the periapical tissues. During photodynamic therapy (PDT), light will excite the drug in bacteria within the root canal but could also potentially affect the periapical host cells that have taken up the drug. Therefore, it is important to establish the safety of PDT and to determine the therapeutic window whereby bacteria can be eliminated but host cells are left intact.

In this study, the viability of human gingival fibroblasts and osteoblasts in vitro was assessed after exposure to methylene blue (MB) and red light with parameters similar to those that may be applied in a clinical setting. Sodium hypochlorite was also tested on gingival fibroblasts and osteoblasts in order to have a reference level for the cytotoxicity of current endodontic chemical agents. Both cell types were sensitized with 50 μg/mL methylene blue followed by exposure to red light at 665 nm for 5 minutes with an irradiance of 10, 20, and 40 mW/cm². The power densities were 10, 20, and 40 mW/cm², corresponding with energy fluencies of 3, 6, and 12 J/cm², respectively. In previous studies, light with a power density of 100 mW/cm² was used for killing endodontic pathogens within infected root canal specimens in vitro. A portion of that light propagates in dentin and escapes from the tooth. The power density of light escaping from the root specimen ranges from 5 to 10 mW/cm² depending on the morphology of the root canal system. In the present study, up to fourfold greater power densities were used.

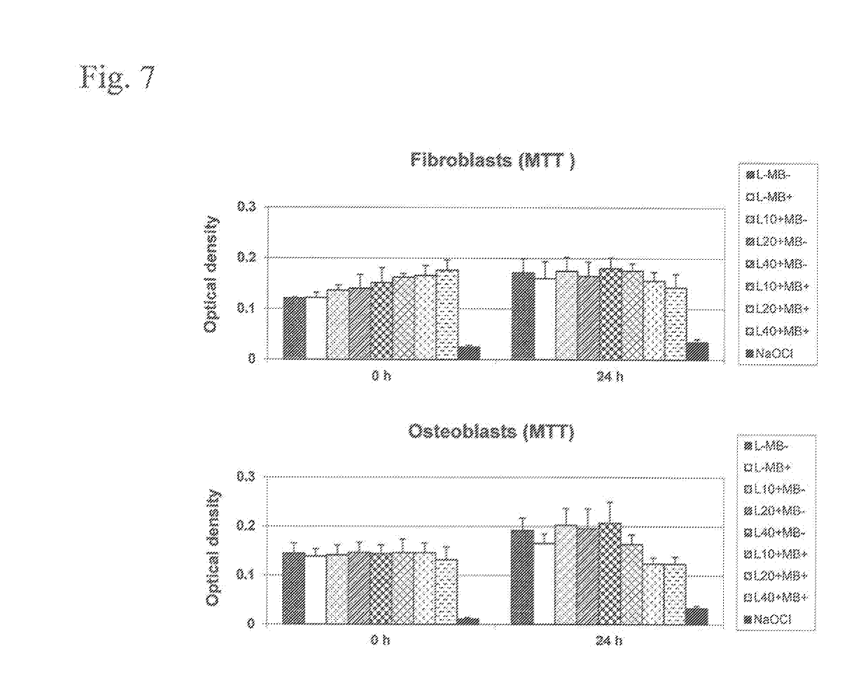

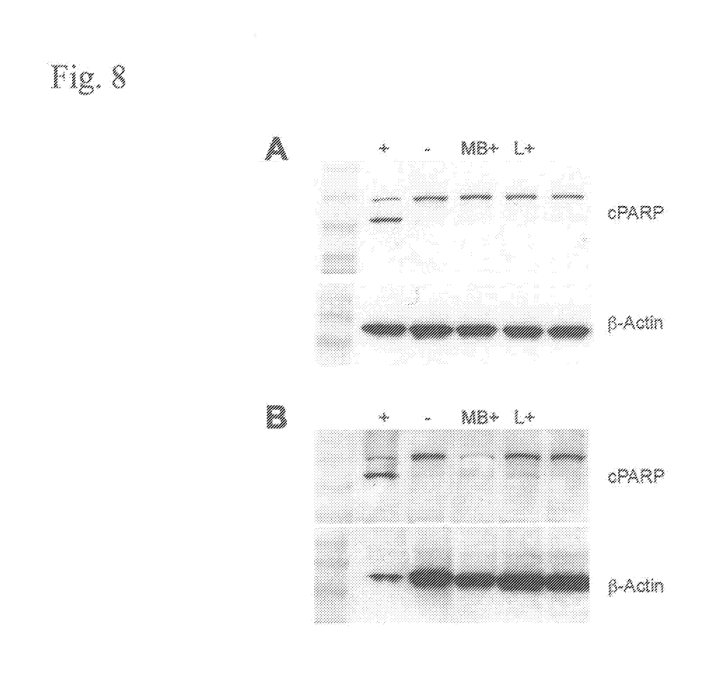

After photodynamic therapy (PDT), cell viability and mitochondrial activity were evaluated by the neutral red and MTT assay, respectively. The assessment of PDT-induced apoptosis was investigated by western blot analysis using cleaved poly(ADPribose) polymerase-specific antibodies. Light at 20 and 40 mW/cm² with MB had modest effects at 24 hours on osteoblasts in both assays, whereas sodium hypochlorite completely eliminated cells. FIG. 7 shows the growth of MB-sensitized gingival fibroblasts and osteoblasts as determined by the MTT assay (assessment of mitochondrial activity) after their exposure to red light. Light alone had no effect on viability and mitochondrial activity of either cell type at 0 and 24 hours. MB alone had no effect on fibroblasts at 0 and 24 hours, whereas it slightly reduced the viability and mitochondrial activity of osteoblasts ranging from 9.4% (0 hours) to 14.6% (24 hours) and from 3.1% (0 hours) to 13.5% (24 hours), respectively. The results for the L+MB+ groups were as follows: For fibroblasts, all three groups that received light and MB (L10+MB+, L20+MB, and L40+MB+) were significantly higher than both L-MB+ and L-MB− at 0 hours. At 24 hours, mitochondrial activity for the L40+MB+ group was reduced by 18.5% and was significantly lower than L40+MB− and control. For osteoblasts, the mean OD levels showed no significant differences among treatment groups at 0 hours. At 24 hours, the mitochondrial activity for the L20+MB+ and L40+MB+ groups was reduced by approximately 34% and was significantly lower than all other treatments. Treatment with sodium hypochlorite (NaOCl) that is used clinically resulted in severe reductions to levels ranging from 0% to 20% of control for mean OD levels of fibroblasts and osteoblasts in both assays at 0 and 24 hours. Western blot analysis revealed no signs of apoptosis in either cell type (FIG. 8). Osteoblasts (A) and fibroblasts (B) were treated with MB only (lane MB+), light only (lane L+), or light and MB (lane L+MB+). Control cultures were left untreated (lane −) or treated with 200 μmol/L staurosporine (lane +). Twenty-four hours after treatment, cultures were stopped and analyzed by western blot using cleaved PARP− and b-actin-specific antibodies. Neither treatment resulted in PARP cleavage. These findings suggest that MB-mediated PDT, as an adjunctive technique for endodontic disinfection, may be effectively used in a clinical setting without harming cells in the periapical region. The current study provides evidence that PDT with light parameters used for bacterial targeting in the root canal system (power density: 100 mW/cm²; energy fluence: 30 J/cm²) displays a safe therapeutic window.

### Example 3

Evaluation of the anti-microbial effects of PDT on infected human teeth ex vivo (Ng R et al (2011). Endodontic photodynamic therapy ex vivo. J. Endod. 37(2): 217-222.)

The objective of this study was to evaluate the antimicrobial effects of photodynamic therapy (PDT) on infected human teeth ex vivo. Fifty-two freshly extracted teeth with pulpal necrosis and associated periradicular radiolucencies were obtained from 34 subjects. Twenty-six teeth with 49 canals received chemomechanical debridement (CMD) with 6% NaOCl, and 26 teeth with 52 canals received CMD plus PDT. For PDT, root canal systems were incubated with methylene blue (MB) at concentration of 50 μg/mL for 5 minutes, followed by exposure to red light at 665 nm (using the fiber in FIG. 2) with an energy fluence of 30 J/cm². The contents of root canals were sampled by flushing the canals at baseline and after CMD alone or CMD+PDT and were serially diluted and cultured on blood agar. Survival fractions were calculated by counting colony-forming units (CFUs). Partial characterization of root canal species at baseline and after CMD alone or CMD+PDT was performed by using DNA probes to a panel of 39 endodontic species in the checkerboard assay. The Mantel-Haenszel χ2 test for treatment effects demonstrated the better performance of CMD+PDT over CMD (P=0.026). CMD+PDT significantly reduced the frequency of positive canals relative to CMD alone (P=0.0003) (Table 2). After CMD+PDT, 45 of 52 canals (86.5%) had no CFUs as compared with 24 of 49 canals (49%) treated with CMD (canal flush samples). The CFU reductions were similar when teeth or canals were treated as independent entities.

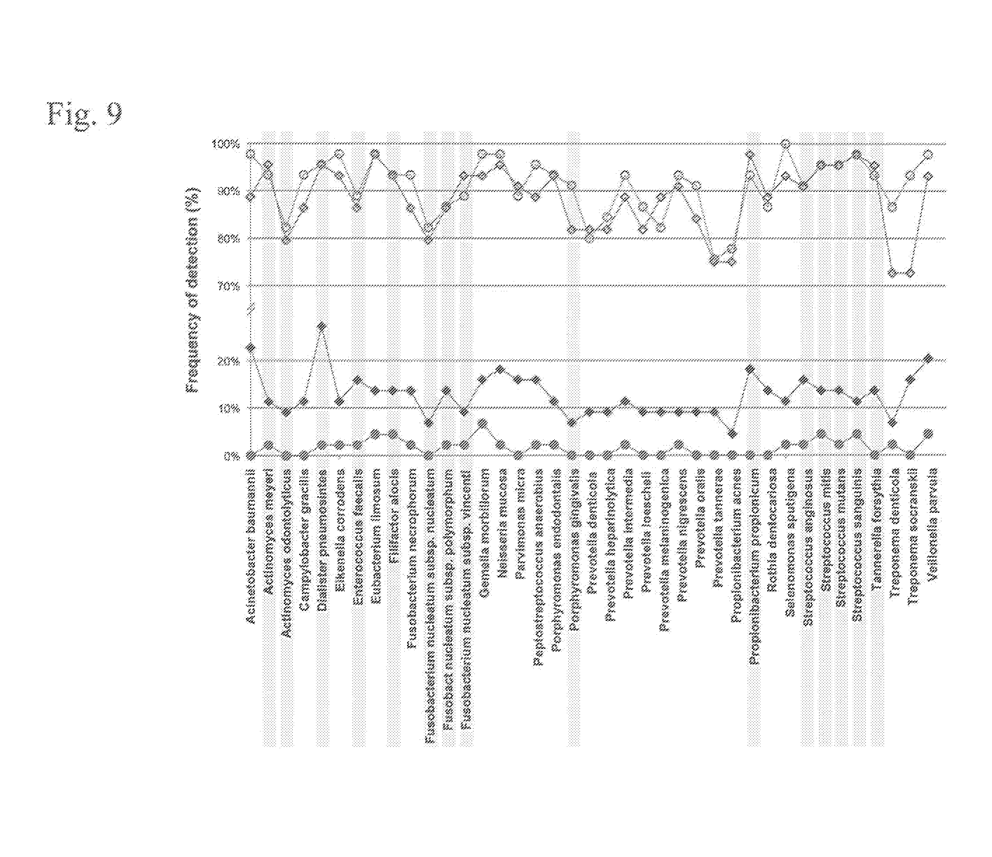

Post-treatment detection levels for all species were markedly lower for canals treated by CMD+PDT than they were for those treated by CMD alone (FIG. 9). Bacterial species within dentinal tubules were detected in 17 of 22 (77.3%) and 15 of 29 (51.7%) canals in the CMD and CMD+PDT groups, respectively (P=0.034) (Table 3). Data indicate that PDT significantly reduces residual bacteria within the root canal system, and that PDT, if further enhanced by technical improvements, holds substantial promise as an adjunct to CMD.

Table 2 presents the frequency of root canal infection following chemo mechanical debridement (CMD) or CMD+photodynamic therapy (PDT) in naturally-infected extracted human teeth:

TABLE 2

| Survival post | | | | Total # of |
|---|---|---|---|---|
| treatment: | None | <0.1% | >0.1% | canals |

| | | | | | |
|---|---|---|---|---|---|
| CMD | # | 24 | 14 | 11 | 49 |
| | % | 49.0 | 28.6 | 22.4 | |
| CMD + PDT | # | 45 | 7 | 0 | 52 |
| | % | 86.5 | 13.5 | 0.0 | |

Table 3 presents the frequency of infection in dentinal tubules following CMD or CMD+PDT.

TABLE 3

| Survival post | | |
|---|---|---|
| treatment: | POSITIVE | NEGATIVE |
| CMD | 17 | 5 |
| CMD + PDT | 15 | 14 |

### EQUIVALENTS

Those skilled in the art will recognize, or be able to ascertain using no more than routine experimentation, numerous equivalents to the devices, systems, kits, and methods of use thereof described herein. Such equivalents are considered to be within the scope of this invention and are covered by the following claims. Those skilled in the art will also recognize that all combinations of embodiments described herein are within the scope of the invention.

What is claimed is:

1. A handheld dental device, comprising:
a. an elongated housing having a distal end and a proximal end;
b. an applicator tip extending from the distal end of the housing, wherein the applicator tip has a lumen defined by a wall;
c. an optic fiber extending from the distal end of the housing through the lumen of the applicator tip;
d. at least one light source within or at the housing, wherein the light source provides light to the optic fiber;
e. at least one power source attached to or residing within the housing, wherein the power source is in communication with at least the light source; and
f. at least one oxygen source within or at the housing,
wherein:
the oxygen source provides oxygen to a passage that communicates with the oxygen source and the applicator tip; and
oxygen and light are provided at the applicator tip.

2. The handheld dental device of claim 1, further comprising a switch that communicates with the light source, the passage of oxygen, or both.

3. The handheld dental device of claim 1, wherein oxygen and light are provided at the applicator tip simultaneously.

4. The handheld dental device of claim 1, wherein the light source emits a wavelength that ranges from about 650 nm to about 1400 nm.

5. The handheld dental device of claim 1, wherein the device emits a power density that ranges from about 1 mW/cm² to about 200 mW/cm².

6. The handheld dental device of claim 1, wherein the device emits an energy fluence that ranges from about 0.1 Joules/cm² to about 1000 Joules/cm².

7. The handheld dental device of claim 1, wherein the optic fiber comprises at least one diffuser for distributing light.

8. The handheld dental device of claim 7, wherein the diffuser distributes light in essentially 360 degrees.

9. The handheld dental device of claim 1, wherein the optic fiber has a diameter ranging from about 100 μm to 500 μm.

10. The handheld dental device of claim 1, wherein the optic fiber has a length ranging from about 5 mm to 40 mm.

11. The handheld dental device of claim 1, wherein the applicator tip comprises a plurality of pores through which the oxygen flows.

12. The handheld dental device of claim 1, wherein the applicator tip is flexible.

13. The handheld dental device of claim 12, wherein the applicator tip bends in a range from about 5 degrees to about 90 degrees.

14. The handheld dental device of claim 1, wherein the applicator tip has a length ranging from about 5 mm to 40 mm.

15. The handheld dental device of claim 1, wherein the oxygen flows from the applicator tip at a rate between 1 ml/min and 10 ml/min.

16. A handheld dental device, comprising:
a. an elongated housing having a distal end and a proximal end;
b. an applicator tip extending from the distal end of the housing, wherein the applicator tip has a lumen defined by a wall;
c. an optic fiber extending from the distal end of the housing through the lumen of the applicator tip;
d. at least one light source within or at the housing, wherein the light source provides light to the optic fiber and emits a wavelength that ranges from about 650 nm to about 1400 nm;
e. at least one power source attached to or residing within the housing, wherein the power source is in communication with at least the light source; and
f. at least one oxygen source within or at the housing,
wherein:
the oxygen source provides oxygen to a passage that communicates with the oxygen source and the applicator tip;
the oxygen and light are provided at the applicator tip simultaneously;
the device emits a power density that ranges from about 1 mW/cm² to about 200 mW/cm²; and
the device emits an energy fluence that ranges from about 0.1 Joules/cm² to about 1000 Joules/cm².

17. A method for providing light and oxygen to a target area of an oral cavity of an individual in need thereof, comprising engaging the handheld dental device of claim 1 to administer light and oxygen to the target area of the oral cavity of the individual.

18. The method of claim 17, further comprises applying at least one photosensitizer to the target area.

19. The method of claim 18, wherein the at least one photosensitizer is selected from the group consisting of methylene blue, acridine orange, porfimer sodium, benzoporphyrin derivative, methoxsalen, psoralen, talaporfin, temoporfin, verteporfin, and combinations thereof.

20. A method for improving oral health by providing light and oxygen to a target area of an oral cavity of an individual in need thereof, comprising administering light and oxygen to the target area of the oral cavity of the individual, wherein:
the light and oxygen are provided using the handheld device of claim 1; and
improving oral health comprises reducing at least one of: a number of one or more bacteria; a number of one or more fungi; the severity or number of endodontic treatment failures; and at least one symptom associated with one or more gum conditions or diseases.

21. A method for reducing one or more bacteria or fungi at a target area of an oral cavity of an individual in need thereof, comprising administering light and oxygen to the target area of the oral cavity of the individual, wherein the light and oxygen is administered to the target area using the handheld device of claim 1.

22. A dental system, comprising:
a. a handheld dental device comprising:
(i) an elongated housing having a distal end and a proximal end;
(ii) an optic fiber extending from the distal end of the housing, wherein the optic fiber is adapted to extend through the lumen of an applicator tip; and
(iii) at least one light source within or at the housing, wherein the light source provides light to the optic fiber;
b. an applicator tip for attachment to the distal end of the housing, wherein the applicator tip has a lumen defined by a wall;
c. at least one power source for attachment or insertion to the housing, wherein the power source is in communication with at least the light source;
d. at least one oxygen source for attachment or insertion to the housing, wherein the oxygen source provides oxygen to a passage that communicates with the oxygen source and the applicator tip; and
e. at least one photosensitizer.

23. The dental system of claim 22, wherein the photosensitizer comprises: methylene blue, acridine orange, porfimer sodium, benzoporphyrin derivative, methoxsalen, psoralen, talaporfin, temoporfin, verteporfin, or a combination thereof.

24. A dental kit, comprising:
a. a handheld dental device comprising:
(i) an elongated housing having a distal end and a proximal end;
(ii) an optic fiber extending from the distal end of the housing, wherein the optic fiber is adapted to extend through the lumen of an applicator tip; and
(iii) at least one light source within or at the housing, wherein the light source provides light to the optic fiber;
b. an applicator tip for attachment to the distal end of the housing, wherein the applicator tip has a lumen defined by a wall;
c. at least one power source for attachment or insertion to the housing, wherein the power source is in communication with at least the light source;
d. at least one oxygen source for attachment or insertion to the housing, wherein the oxygen source provides oxygen to a passage that communicates with the oxygen source and the applicator tip; and
e. at least one agent that assists in eliminating bacteria, cleaning teeth, tongue or gums, or any combination thereof.

25. The dental kit of claim 24, wherein the agent comprises photosensitizer.

26. The dental kit of claim 25, wherein the photosensitizer comprises: methylene blue, acridine orange, porfimer sodium, benzoporphyrin derivative, methoxsalen, psoralen, talaporfin, temoporfin, verteporfin, or a combination thereof.
